(19) United States
(12) Patent Application Publication
(10) Pub. No.: US 2026/0218259 A1
(43) Pub. Date: Jul. 30, 2026

(54) BIOLOGICAL FLOCCULANT, PREPARATION METHOD THEREFOR AND USE THEREOF IN CONTROL OF CYANOBACTERIA

(71) Applicant: SOUTH CHINA UNIVERSITY OF TECHNOLOGY, Guangzhou, Guangdong (CN)

(72) Inventors: Jinxia Zhou, Guangzhou, Guangdong (CN); Hao Liu, Guangzhou, Guangdong (CN)

(21) Appl. No.: 19/145,619

(22) PCT Filed: Dec. 2, 2022
(86) PCT No.: PCT/CN2022/136394

§ 371 Date: Jul. 3, 2025

(30) Foreign Application Priority Data
May 7, 2022 (CN) 202210493531.3

(51) Int. Cl. C12P 19/04; C02F 1/56; C12N 1/205; C12R 1/01

(52) U.S. Cl. CPC C12P 19/04; C02F 1/56; C12N 1/205; C12R 2001/01

(57) ABSTRACT

Provided are a biological flocculant, a preparation method therefor and use thereof in control of Cyanobacteria. The biological flocculant includes cationic hydroxyethyl cellulose and an extracellular polysaccharide. The extracellular polysaccharide is produced by fermentation of Agrobacterium sp. LHZJX.A07 that is taxonomically named. A method for preparing the extracellular polysaccharide includes screening Agrobacterium from a soil enrichment culture dilution spread plate, purifying and inoculating the Agrobacterium into a culture medium for culture, performing fermentation to obtain a extracellular polysaccharide crude solution, and performing concentration, purification and drying to obtain the extracellular polysaccharide.

### CROSS-REFERENCE TO RELATED APPLICATION

This application is a national stage application of PCT application serial no. PCT/CN2022/136394, filed on Dec. 2, 2022, which claims the priority of Chinese patent application No. 202210493531.3, filed on May 7, 2022. The entirety of each of the above-mentioned patent applications is hereby incorporated by reference herein and made a part of this specification.

### REFERENCE TO SEQUENCE LISTING

This application includes a Sequence Listing filed electronically as an XML file named “P22035HG-seql.xml”, created on Dec. 1, 2022, with a size of 3,275 bytes. The Sequence Listing is incorporated herein by reference.

### TECHNICAL FIELD

The present invention relates to the fields of microbiology, bioengineering fermentation technology and environment, and specifically relates to a biological flocculant, a preparation method therefor and use thereof in control of Cyanobacteria.

### BACKGROUND

Cyanobacteria, also known as blue-green algae, are one kind of most primitive and oldest algae plants. The Cyanobacteria are widely distributed throughout the world. In some nutrient-rich water bodies, some Cyanobacteria usually have mass propagation in summer and form a layer of blue-green fishy foam, called “water blooms”, on a water surface. A large-scale outbreak of the Cyanobacteria not only will cause deterioration of water quality and depletion of oxygen in water in severe cases to result in fish death, but also will change community structures of lakes to reduce biodiversity.

The outbreak of algal blooms is closely related to eutrophication of water bodies. At present, about 60% of lakes all over the world are in varying degrees of eutrophication states. In China, among 107 freshwater lakes monitored in different regions in 2018, proportions of eutrophic lakes, mesotrophic lakes and oligotrophic lakes are 29%, 61.7% and 0.3%, respectively. Therefore, control of the Cyanobacteria becomes urgent. Due to distribution in water, the Cyanobacteria need to be flocculated into clusters for better salvage. The patent “Mixed Flocculant and Method for Controlling Cyanobacterial Blooms Using Same” discloses a mixed flocculant and a method for controlling cyanobacterial blooms using the same. The mixed flocculant is obtained by mixing grade-three sepiolite velvet, talc and diatomite. The mixed flocculant has a low cost and a good flocculation effect, but has certain harm to the environment since too much talc is used in the components.

The patent “Method for Controlling Cyanobacteria Using Microbial Flocculant and Hydrolyzed Salt” discloses a method for controlling Cyanobacteria using a microbial flocculant and a hydrolyzed salt. According to the method, the microbial flocculant prepared from Bacillus mucilaginosus and the hydrolyzed salt are added into a Cyanobacteria outbreak water body, and the microbial flocculant and the hydrolytic salt have a synergistic effect to flocculate Cyanobacteria and make a Cyanobacteria flocculate float on a liquid surface. A flocculation effect is good, but metal ions used in the method are not environmentally friendly.

### SUMMARY

An objective of the present invention is to propose a biological flocculant and a preparation method therefor. The flocculant is prepared by using cationic hydroxyethyl cellulose and an extracellular polysaccharide, and the two both have the properties of being natural, non-toxic and degradable and are environmentally friendly.

Another objective of the present invention is to propose use of the above biological flocculant in control of Cyanobacteria, which can flocculate dispersed Cyanobacteria and can destroy Cyanobacteria cells while inhibiting the activity of the Cyanobacteria without releasing Cyanobacteria toxins to purify a water body.

In the present invention, a strain of Agrobacterium for producing the extracellular polysaccharide is obtained by screening from soil, which is taxonomically named as Agrobacterium sp. LHZJX.A07 and has been preserved in Guangdong Microbial Culture Collection Center with a accession number GDMCC NO: 62125 on a preservation date of Mar. 17, 2022. An address of a preservation department is: Institute of Microbiology, Guangdong Academy of Sciences, 5th Floor, No. 59 Building, No. 100 Courtyard, Martyr Middle Road, Guangzhou, post code: 510070.

The present invention proposes that the screened Agrobacterium is used for fermentation of the extracellular polysaccharide to prepare the biological flocculant.

The objectives of the present invention are achieved through the following technical solutions.

A biological flocculant includes cationic hydroxyethyl cellulose and an extracellular polysaccharide. The extracellular polysaccharide is produced by fermentation of Agrobacterium sp. LHZJX.A07 that is taxonomically named, and the strain has been preserved in Guangdong Microbial Culture Collection Center with a accession number GDMCC NO: 62125 on a preservation date of Mar. 17, 2022.

A method for preparing the biological flocculant includes the following steps:
(1) isolating and screening an Agrobacterium strain from soil, purifying and inoculating the strain into a slant culture medium for storage; rinsing off bacterial moss on a slant with sterile water under sterile conditions, and inoculating a bacterial solution into a seed culture medium; and performing culture at 30-40° C. and 125-150 r/min for 12-24 h;
(2) inoculating a seed solution cultured in the step (1) into a fermentation culture medium for culture according to an inoculation amount of 3%-6% and performing culture at 30-40° C. and 120-150 r/min for 1-5 d;
(3) subjecting a fermentation solution in the step (2) to high speed centrifugation at 1×10⁴-1.6×10⁴ r/min, and collecting a supernatant to obtain a extracellular polysaccharide crude solution;
(4) subjecting the extracellular polysaccharide crude solution obtained in the step (3) to concentration, purification and drying to obtain an extracellular polysaccharide;
(5) first dispersing 10-30 g of hydroxyethyl cellulose in 100 mL of a mixture of isopropanol and water, performing stirring evenly, then adding 3-8 mL of a 25 wt % NaOH solution, and finally adding 3-10 mL of a 65 wt % 3-chloro-2-hydroxypropyltrimethylammonium chloride solution to carry out a reaction at 60-70° C. for 2-8 h; and after the reaction completed, performing purification with acetone (analytical pure), and performing washing, freeze drying and sieving with a 60-100 mesh sieve to obtain cationic hydroxyethyl cellulose; and
(6) evenly stirring the cationic hydroxyethyl cellulose prepared in the step (5), then adding the Agrobacterium extracellular polysaccharide prepared in the step (4), performing stirring evenly, and then performing standing to obtain the biological flocculant.

Further, a formulation of the seed culture medium in the step (1) includes: sucrose (4-6 g/L), Na₂HPO₄ (1-4 g/L), MgSO₄·7H₂O (0.2-1 g/L), CaCO₃ (0.05-0.15 g/L) and FeCl₃ (0.001-0.01 g/L), and has a pH of 6.5-7.5.

Further, a formulation of the fermentation culture medium in the step (2) includes: KH₂PO₄ (0.1-0.5 g/L), MgSO₄·7H₂O (0.1-0.5 g/L), CaCO₃ (2-5 g/L), CaSO₄ (0.05-0.2 g/L), NaCl (0.1-0.5 g/L) and mannitol (8-10 g/L), and has a pH of 6.8-7.0.

Preferably, the formulation of the seed culture medium in the step (1) includes: the sucrose (5 g/L), the Na₂HPO₄ (2 g/L), the MgSO₄·7H₂O (0.5 g/L), the CaCO₃ (0.1 g/L) and the FeCl₃ (0.005 g/L), and has the pH of 7.0.

Preferably, the formulation of the fermentation culture medium in the step (2) includes: the KH₂PO₄ (0.2 g/L), the MgSO₄·7H₂O (0.2 g/L), the CaCO₃ (5 g/L), the CaSO₄ (0.1 g/L), the NaCl (0.2 g/L) and the mannitol (10 g/L), and has the pH of 6.8.

Preferably, the inoculation amount in the step (2) is 4%, and a shake culture time is 5 d.

Preferably, a rotational speed of the high speed centrifugation in the step (3) is 1.2×10⁴ r/min, and a centrifugation time is 10 min.

Further, the subjecting the extracellular polysaccharide crude solution to concentration, purification and drying to obtain an extracellular polysaccharide in the step (4) specifically includes the following steps:
(a) subjecting the extracellular polysaccharide crude solution to concentration by using a dialysis bag to obtain an extracellular polysaccharide concentrate, where a cut-off amount of the dialysis bag used is 3-8 kDa (kilodalton), and a dialysis time is 48-72 h; and adding the extracellular polysaccharide concentrate into ethanol (where a mass fraction of the ethanol is 95%-100%, and a volume ratio of the extracellular polysaccharide concentrate to the ethanol is 1:2 to 1:6), performing stirring at a constant speed by using a magnetic stirrer for 5-10 min, performing standing for 12-24 h, performing centrifugation at 1×10⁴-1.6×10⁴ r/min, removing a supernatant, and collecting a precipitate;
(b) adding pure water that is ⅓ of the volume of the extracellular polysaccharide concentrate in the step (a) into the precipitate collected in the step (a), performing dissolution in a water bath at 37-45° C. for 20-60 min, and performing centrifugation to collect a supernatant; and
(c) adding the supernatant collected in the step (b) into ethanol (wherein a mass fraction of the ethanol is 95%-100%, and a volume ratio of the supernatant to the ethanol is 1:2 to 1:6), performing stirring at a constant speed by using a magnetic stirrer for 5-10 min, performing standing for 12-24 h, performing centrifugation at 1×10⁴-1.6×10⁴ r/min, removing a supernatant, collecting a precipitate, and performing freeze drying at 0-4° C. and sieving with a 60-100 mesh sieve to obtain the Agrobacterium extracellular polysaccharide.

Preferably, the ethanol in the step (a) and the step (c) is 95% ethanol; the volume ratio of the extracellular polysaccharide concentrate to the ethanol in the step (a) is 1:3; and the volume ratio of the supernatant to the ethanol in the step (c) is 1:3.

Preferably, a mesh number of the sieving in the step (c) is 80 mesh.

A formulation of the biological flocculant of the present invention includes: 0.20-1 g/L of the cationic hydroxyethyl cellulose prepared by the above method and 20-400 mg/L of the Agrobacterium extracellular polysaccharide.

Further, an optimal formulation of the biological flocculant includes: 20 mg/L of the cationic hydroxyethyl cellulose and 177 mg/L of the Agrobacterium extracellular polysaccharide.

The present invention provides use of the biological flocculant prepared by the above method in control of Cyanobacteria.

### BENEFICIAL EFFECTS OF THE INVENTION

Compared with the prior art, beneficial effects of the present invention are shown as follows.
(1) The biological flocculant can be prepared by using the self-fermented extracellular polysaccharide and the self-prepared cationic hydroxyethyl cellulose in the present invention, and the obtained biological flocculant can flocculate the Cyanobacteria dispersed in a water body and has the effect of inhibiting the growth of the Cyanobacteria without releasing toxins from the suspended Cyanobacteria.
(2) The Cyanobacteria flocculant is prepared by using the extracellular polysaccharide and the cationic hydroxyethyl cellulose in the present invention, and the two are both a natural, low-toxic and degradable polysaccharide and have no harm to water environment.
(3) When the biological flocculant of the present invention is used in control of the Cyanobacteria, a flocculation rate of the Cyanobacteria is as high as 99.8%. Meanwhile, a chlorophyll clearance rate of Microcystis aeruginosa reaches 73%.

### BRIEF DESCRIPTION OF DRAWINGS

FIG. 1 shows a Gram staining microscope photo of an Agrobacterium strain screened from soil in the present invention;

FIG. 2 shows a phylogenetic tree diagram constructed based on a 16S rDNA sequence of an Agrobacterium strain screened from soil in the present invention;

FIG. 3 shows a GPC diagram of an Agrobacterium extracellular polysaccharide obtained in Example 1;

FIG. 4 shows a GPC diagram of an Agrobacterium extracellular polysaccharide obtained in Example 2;

FIG. 5 shows a GPC diagram of an Agrobacterium extracellular polysaccharide obtained in Example 3;

FIG. 6 shows safety evaluation results of an Agrobacterium extracellular polysaccharide and cationic hydroxyethyl cellulose;

FIG. 7 shows chlorophyll clearance rate results of Microcystis aeruginosa using cationic hydroxyethyl cellulose;

FIG. 8 shows flocculation rate results of Cyanobacteria at different contents of Agrobacterium extracellular polysaccharides when a concentration of cationic hydroxyethyl cellulose is 250 mg/L; and

FIG. 9 shows flocculation rate results of Cyanobacteria at different contents of cationic hydroxyethyl cellulose when a concentration of an Agrobacterium extracellular polysaccharide is 177 mg/L.

### EMBODIMENTS OF THE PRESENT INVENTION

The present invention is further described below in combination with specific examples, but the implementation and scope of protection of the present invention are not limited thereto.

### 1. Isolation and Screening of Strains:

(1) Bacteria enrichment: 1 g of soil was selected from South China University of Technology, added into a nutrient broth culture medium (liquid loading amount: 70 mL culture medium/300 mL triangular flask), heated in a water bath at 80° C. for 20 min, and subjected to shake culture on a shaker for 24 h (at 30° C. and 150 r/min).

(2) Preliminary screening: 1 mL of an enrichment solution in the step (1) was selected and added into 9 mL of sterile water to obtain a 10⁻¹ sample diluent, and different gradients, such as 10⁻¹, 10⁻², 10⁻³, . . . , 10⁻⁹ and 10⁻¹⁰, of diluents were sequentially obtained according to the method. 0.1 mL of various gradients of the diluents were coated onto silicate plates and cultured under constant-temperature conditions at 37° C. for 24 h, respectively.

(3) Purification: Viscous and transparent strains were selected to the corresponding plates, diluted and coated to obtain individual bacterial colonies; and the individual bacterial colonies on the plates were inoculated into a slant culture medium for storage.

(4) Secondary screening: The strains obtained after isolation and purification were inoculated into a silicate liquid culture medium (liquid loading amount: 70 mL culture medium/300 mL triangular flask), and the culture medium was shaken on a shaker for fermentation under constant-temperature conditions at 37° C. for 72 h. When the viscosity of the culture medium was increased, contents of extracellular polysaccharides were determined by adopting an ethanol precipitation method.

A specific implementation method of the ethanol precipitation method is as follows: A fermentation solution with increased viscosity in the step (3) was selected and subjected to centrifugation at 1.2×10³ r/min for 10 min, a supernatant was selected, added into 95% ethanol (V:V=1:3) and mixed evenly by using an oscillator, a fermentation solution-ethanol mixture was subjected to centrifugation at 1.2×10³ r/min for 10 min, a precipitate was collected, added into hot water for dissolution and then subjected to centrifugation, a supernatant was selected for secondary precipitation with ethanol, centrifugation was performed to collect a precipitate, and the precipitate was subjected to drying, grinding and sieving with a 80 mesh sieve.

### 2. Identification and Recognition of Strains:

### (1) Gram Staining

Slice preparation: 5 mL of normal saline was selected, injected into a slant test tube of Agrobacterium and gently shaken evenly, one drop of a bacterial solution was dropped on a slide, and a specimen surface of the slide was placed upward and slowly dried with far fire of an alcohol lamp; and a back surface of the slide coated with a specimen was allowed to pass through a place with the highest flame temperature of the alcohol lamp for 3 times at a pendulum speed to fix bacteria on the slide.

Staining: (Primary staining) A few drops of a crystal violet staining solution were dropped on the fixed bacteria coated slice for action at room temperature for 1 min, and then the slice was gently rinsed with a trickle of water; (mordanting) a few drops of an iodine solution as a mordant were dropped for action at room temperature for 1 min, and then the slice was rinsed with a trickle of water; (decolorization) a few drops of 95% alcohol were dropped, the slide was gently shaken for a few seconds for even decolorization, then the slide was slanted to make a decolorized dye flow away with the alcohol, then alcohol was dropped until the flowing alcohol was colorless or slightly lilac (for about 30 s), and the alcohol was rinsed off immediately with a trickle of water; and (secondary staining) after a sand yellow staining solution was dropped for secondary staining and action at room temperature for 1 min, the slice was gently rinsed with a trickle of water.

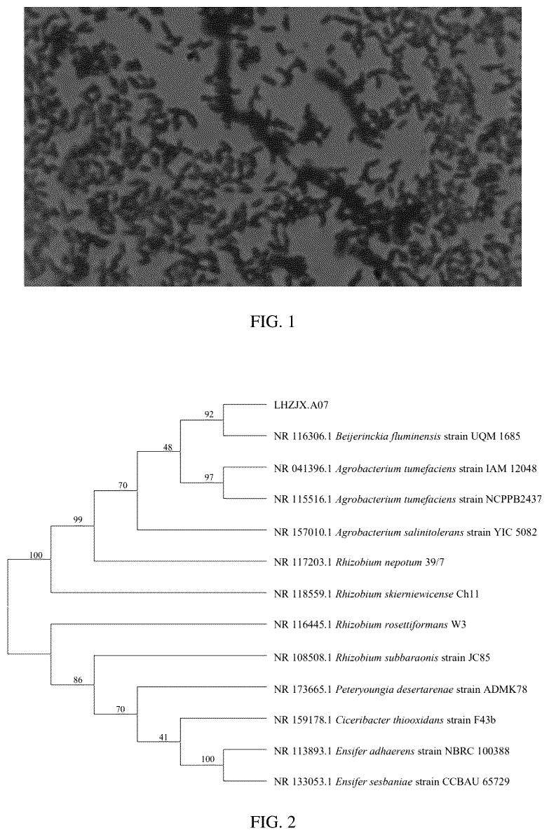

Microscopic examination: A specimen was air-dried after staining. According to observation under a microscope, Gram-positive bacteria were purple, and Gram-negative bacteria were red. The strain of the present invention was purple after Gram staining (as shown in FIG. 1), which was a Gram-positive bacterium and was rod-shaped.

(2) 16S rDNA Gene Sequencing

A bacterial genomic DNA extraction kit was used to extract DNA of the strain obtained after the secondary staining, and 16S rRNA was amplified by using an upstream primer 27F and a downstream primer 1492R PCR. A product obtained after amplification was subjected to gel recovery and purification and then sent to Guangzhou IGE Biotechnology Co., Ltd. for gene sequencing.

Sequencing results show that a 16S rRNA gene of the strain has a length of 1341 bp (see SEQ ID NO: 1). According to the comparison between the sequencing results and an NCBI database, known sequences with highest homology with a 16S rDNA sequence of the strain can be obtained. A phylogenetic tree diagram (as shown in FIG. 2) of the strain was constructed according to similarity analysis results of the 16S rDNA sequence. Through the comparison, it can be seen that the strain has highest similarity with Agrobacterium sp., and a matching degree reaches 95.78%. Therefore, it can be concluded that the strain isolated and screened in the present invention is Agrobacterium, which is specifically named as Agrobacterium sp. LHZJX.A07. The strain has been preserved in Guangdong Microbial Culture Collection Center with an accession number GDMCC NO: 62125 on a preservation date of Mar. 17, 2022.

### Example 1

### I. Preparation of an Agrobacterium Extracellular Polysaccharide:

(1) Preparation of a seed culture medium: The seed culture medium was formulated from sucrose (5 g/L), Na₂HPO₄ (2 g/L), MgSO₄·7H₂O (0.5 g/L), CaCO₃ (0.1 g/L) and FeCl₃ (0.005 g/L), and had a pH of 7.0.

Preparation of a fermentation culture medium: The fermentation culture medium was formulated from KH₂PO₄ (0.2 g/L), MgSO₄·7H₂O (0.2 g/L), CaCO₃ (5 g/L), CaSO₄ (0.1 g/L), NaCl (0.2 g/L) and mannitol (10 g/L), and had a pH of 6.8. The culture media were separately loaded into 3 pieces of 300 mL triangular flasks with a liquid loading amount of 25%. Sterilization was performed at 121° C. for 20 min.

(2) Bacterial activation: Agrobacterium slants were taken out from a refrigerator at 4° C. and placed in an incubator for activation at 28° C. for 2 h.

(3) Liquid fermentation: 2 bacterial slants in the step (2) were selected, bacterial moss on each slant was rinsed off with 5 mL of sterile water under sterile conditions, and a bacterial solution was inoculated into the seed culture medium prepared in the step (1) and subjected to shake culture at 135 r/min and 37° C. for 2 d to obtain a seed solution; and the seed solution was inoculated into the fermentation culture medium prepared in the step (1) for shake culture with an inoculation amount of 4% at 130 r/min and 37° C. for 3 d.

(4) Extraction of an Agrobacterium extracellular polysaccharide: A fermentation solution in the step (3) was subjected to high speed centrifugation at 1×10⁴ r/min for 10 min, and a supernatant was collected to obtain a extracellular polysaccharide crude solution; a) the extracellular polysaccharide crude solution was subjected to concentration by using a dialysis bag to obtain an extracellular polysaccharide concentrate; and the extracellular polysaccharide concentrate was added into ethanol (V:V=1:3), stirring was performed at a constant speed by using a magnetic stirrer for 8 min, standing was performed for 15 h, centrifugation was performed at 1×10⁴ r/min, a supernatant was removed, and a precipitate was collected; b) pure water that was ⅓ of the volume of the extracellular polysaccharide concentrate in the step a) was added into the precipitate collected in the step a), dissolution was performed in a water bath at 37° C. for 30 min, and centrifugation was performed to collect a supernatant; and c) the supernatant collected in the step b) was added into ethanol (V:V=1:3), stirring was performed at a constant speed by using a magnetic stirrer for 8 min, standing was performed for 15 h, centrifugation was performed at 1×10⁴ r/min, a supernatant was removed, and a precipitate was collected to obtain the Agrobacterium extracellular polysaccharide.

### 2. Preparation of Cationic Hydroxyethyl Cellulose:

(1) 10 g of hydroxyethyl cellulose was dispersed in a three-necked flask loaded with 100 mL of a mixture of isopropanol and water (V:V=95:5) and stirred evenly, and then 3 ml of a 25 wt % NaOH solution and 8 ml of a 65 wt % 3-chloro-2-hydroxypropyltrimethylammonium chloride solution were added to carry out a reaction at 65° C. for 2 h.

(2) After the reaction was completed, cationic hydroxyethyl cellulose prepared in the step (1) was subjected to purification with acetone (analytical pure), washing, freeze drying, grinding and sieving with an 80 mesh sieve.

3. The prepared cationic hydroxyethyl cellulose was evenly stirred, then the prepared Agrobacterium extracellular polysaccharide was added and evenly stirred, and then standing was performed to obtain a biological flocculant.

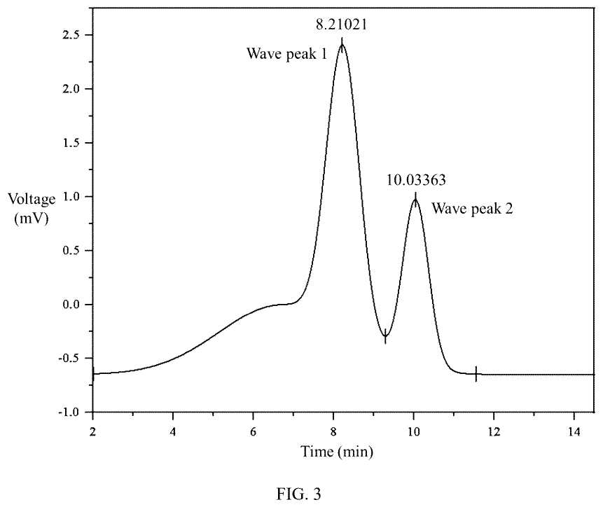

The extracellular polysaccharide obtained in the present example was detected by GPC liquid gel chromatography (as shown in FIG. 3), which mainly had two wave peaks, where an average molecular weight of a wave peak 1 was 246562 (main wave peak), and an average molecular weight of a wave peak 2 was 580.

### Example 2

### I. Preparation of an Agrobacterium Extracellular Polysaccharide:

(1) Preparation of a seed culture medium: The seed culture medium was formulated from sucrose (4 g/L), Na₂HPO₄ (1 g/L), MgSO₄·7H₂O (0.2 g/L), CaCO₃ (0.05 g/L) and FeCl₃ (0.001 g/L), and had a pH of 6.5.

Preparation of a fermentation culture medium: The fermentation culture medium was formulated from KH₂PO₄ (0.1 g/L), MgSO₄·7H₂O (0.1 g/L), CaCO₃ (2 g/L), CaSO₄ (0.05 g/L), NaCl (0.1 g/L) and mannitol (8 g/L), and had a pH of 6.9. The culture media were separately loaded into 3 pieces of 300 mL triangular flasks with a liquid loading amount of 25%. Sterilization was performed at 121° C. for 20 min.

(2) Bacterial activation: Agrobacterium slants were taken out from a refrigerator at 4° C. and placed in an incubator for activation at 28° C. for 2 h.

(3) Liquid fermentation: 2 bacterial slants in the step (2) were selected, bacterial moss on each slant was rinsed off with 5 mL of sterile water under sterile conditions, and a bacterial solution was inoculated into the seed culture medium prepared in the step (1) and subjected to shake culture at 125 r/min and 37° C. for 2 d to obtain a seed solution; and the seed solution was inoculated into the fermentation culture medium prepared in the step (1) for shake culture with an inoculation amount of 3% at 120 r/min and 30° C. for 5 d.

(4) Extraction of an Agrobacterium extracellular polysaccharide: A fermentation solution in the step (3) was subjected to high speed centrifugation at 1.2×10⁴ r/min for 15 min, and a supernatant was collected to obtain a extracellular polysaccharide crude solution; a) the extracellular polysaccharide crude solution was subjected to concentration by using a dialysis bag to obtain an extracellular polysaccharide concentrate; and the extracellular polysaccharide concentrate was added into ethanol (V:V=1:6), stirring was performed at a constant speed by using a magnetic stirrer for 10 min, standing was performed for 24 h, centrifugation was performed at 1.2×10⁴ r/min, a supernatant was removed, and a precipitate was collected; b) pure water that was ⅓ of the volume of the extracellular polysaccharide concentrate in the step a) was added into the precipitate collected in the step a), dissolution was performed in a water bath at 40° C. for 20 min, and centrifugation was performed to collect a supernatant; and c) the supernatant collected in the step b) was added into ethanol (V:V=1:6), stirring was performed at a constant speed by using a magnetic stirrer for 10 min, standing was performed for 24 h, centrifugation was performed at 1.2×10⁴ r/min, a supernatant was removed, and a precipitate was collected to obtain the Agrobacterium extracellular polysaccharide.

2. Preparation of cationic hydroxyethyl cellulose:

(1) 20 g of hydroxyethyl cellulose was dispersed in a three-necked flask loaded with 100 mL of a mixture of isopropanol and water (V:V=95:5) and stirred evenly, and then 8 ml of a 25 wt % NaOH solution and 3 ml of a 65 wt % 3-chloro-2-hydroxypropyltrimethylammonium chloride solution were added to carry out a reaction at 60° C. for 6 h.

(2) After the reaction was completed, cationic hydroxyethyl cellulose prepared in the step (1) was subjected to purification with acetone (analytical pure), washing, freeze drying, grinding and sieving with a 60 mesh sieve.

3. The prepared cationic hydroxyethyl cellulose was evenly stirred, then the prepared Agrobacterium extracellular polysaccharide was added and evenly stirred, and then standing was performed to obtain a biological flocculant.

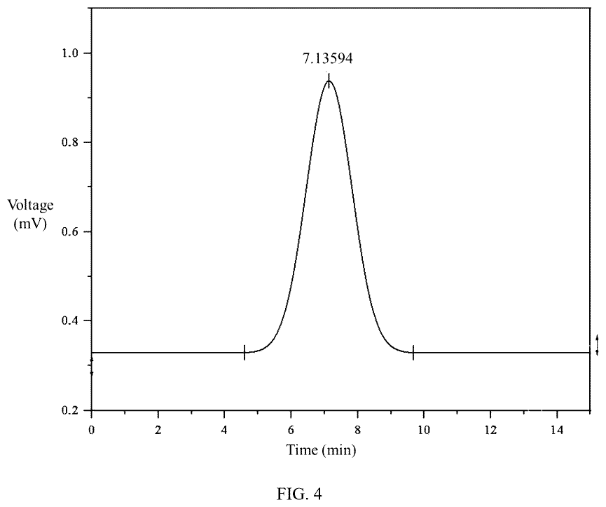

The extracellular polysaccharide obtained in the present example was detected by GPC liquid gel chromatography (as shown in FIG. 4), which mainly had one wave peak after fitting treatment, wherein an average molecular weight of the wave peak was 2682798.

### Example 3

### I. Preparation of an Agrobacterium Extracellular Polysaccharide:

(1) Preparation of a seed culture medium: The seed culture medium was formulated from sucrose (6 g/L), Na₂HPO₄ (4 g/L), MgSO₄·7H₂O (1 g/L), CaCO₃ (0.15 g/L) and FeCl₃ (0.01 g/L), and had a pH of 7.5.

Preparation of a fermentation culture medium: The fermentation culture medium was formulated from KH₂PO₄ (0.5 g/L), MgSO₄·7H₂O (0.5 g/L), CaCO₃ (3 g/L), CaSO₄ (0.2 g/L), NaCl (0.5 g/L) and mannitol (8 g/L), and had a pH of 6.8. The culture media were separately loaded into 3 pieces of 300 mL triangular flasks with a liquid loading amount of 25%. Sterilization was performed at 121° C. for 20 min.

(2) Bacterial activation: Agrobacterium slants were taken out from a refrigerator at 4° C. and placed in an incubator for activation at 28° C. for 2 h.

(3) Liquid fermentation: 2 bacterial slants in the step (2) were selected, bacterial moss on each slant was rinsed off with 5 mL of sterile water under sterile conditions, and a bacterial solution was inoculated into the seed culture medium prepared in the step (1) and subjected to shake culture at 150 r/min and 37° C. for 2 d to obtain a seed solution; and the seed solution was inoculated into the fermentation culture medium prepared in the step (1) for shake culture with an inoculation amount of 6% at 150 r/min and 40° C. for 1 d.

(4) Extraction of an Agrobacterium extracellular polysaccharide: A fermentation solution in the step (3) was subjected to high speed centrifugation at 1.6×10⁴ r/min for 8 min, and a supernatant was collected to obtain a extracellular polysaccharide crude solution; a) the extracellular polysaccharide crude solution was subjected to concentration by using a dialysis bag to obtain an extracellular polysaccharide concentrate; and the extracellular polysaccharide concentrate was added into ethanol (V:V=1:2), stirring was performed at a constant speed by using a magnetic stirrer for 5 min, standing was performed for 12 h, centrifugation was performed at 1.6×10⁴ r/min, a supernatant was removed, and a precipitate was collected; b) pure water that was ⅓ of the volume of the extracellular polysaccharide concentrate in the step a) was added into the precipitate collected in the step a), dissolution was performed in a water bath at 45° C. for 60 min, and centrifugation was performed to collect a supernatant; and c) the supernatant collected in the step b) was added into ethanol (V:V=1:2), stirring was performed at a constant speed by using a magnetic stirrer for 5 min, standing was performed for 12 h, centrifugation was performed at 1.6×10⁴ r/min, a supernatant was removed, and a precipitate was collected to obtain the Agrobacterium extracellular polysaccharide.

### 2. Preparation of Cationic Hydroxyethyl Cellulose:

(1) 30 g of hydroxyethyl cellulose was dispersed in a three-necked flask loaded with 100 mL of a mixture of isopropanol and water (V:V=95:5) and stirred evenly, and then 5 ml of a 25 wt % NaOH solution and 10 ml of a 65 wt % 3-chloro-2-hydroxypropyltrimethylammonium chloride solution were added to carry out a reaction at 70° C. for 8 h.

(2) After the reaction was completed, cationic hydroxyethyl cellulose prepared in the step (1) was subjected to purification with acetone (analytical pure), washing, freeze drying, grinding and sieving with a 100 mesh sieve.

3. The prepared cationic hydroxyethyl cellulose was evenly stirred, then the prepared Agrobacterium extracellular polysaccharide was added and evenly stirred, and then standing was performed to obtain a biological flocculant.

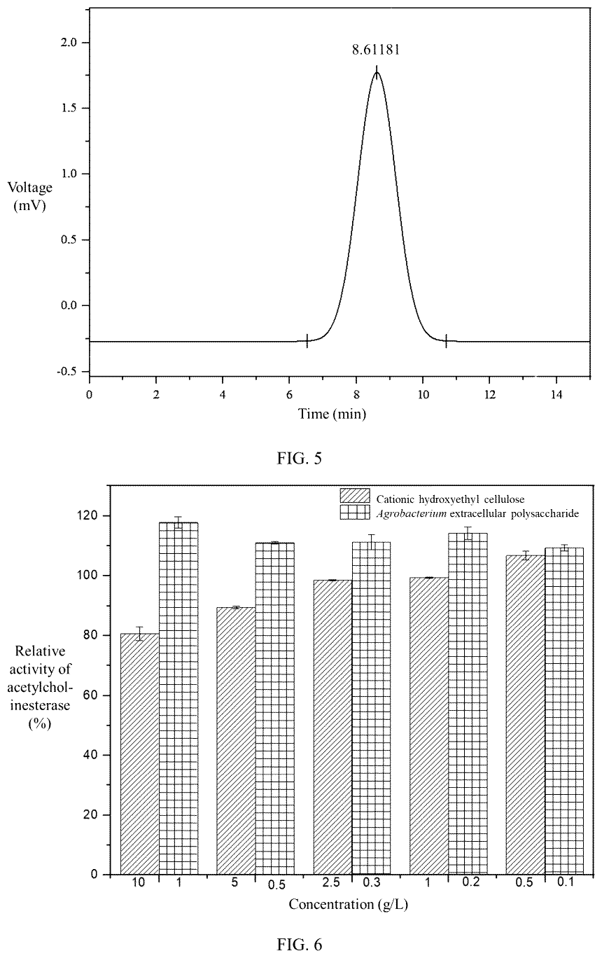

The extracellular polysaccharide obtained in the present example was detected by (YOGO-25010-USPT/02251666v 1) 14 GPC liquid gel chromatography (as shown in FIG. 5), which mainly had one wave peak after fitting treatment, wherein an average molecular weight of the wave peak was 79226.

Evaluation of the safety of cationic hydroxyethyl cellulose and an Agrobacterium extracellular polysaccharide

An absorbance test was carried out on the Agrobacterium extracellular polysaccharide and the cationic hydroxyethyl cellulose prepared in Examples 1-3. The toxicity of the Agrobacterium extracellular polysaccharide and the cationic hydroxyethyl cellulose was evaluated by measuring the decrease of the activity of acetylcholinesterase (AchE). 3 mL of the cationic hydroxyethyl cellulose (0.5-10 g/L) and the Agrobacterium extracellular polysaccharide (0.1-1 g/L) were dissolved in a phosphate buffer solution, and 0.1 mL of a 5,5′-dithio-2-nitrobenzoic acid (DTNB)-phosphoric acid ethanol reagent and 0.1 mL of cholinesterase were transferred into a colorimetric tube. After incubation was performed for 10 min, 0.1 mL of acetylthiocholine iodide was added to start a reaction. The change rate (CR) of the absorbance at 412 nm was determined. A calculation formula of the relative activity (RA) of AchE is as follows:

$${RA}_{AchE} ( \% ) = \frac{{CR}_{us} - {CR}_{ts}}{{CR}_{us}} \times 100 \%$$

In the formula, RA_{AchE} is the relative activity of acetylcholinesterase; CRᵤₛ is the change rate of the absorbance of acetylcholinesterase at 412 nm when the cationic hydroxyethyl cellulose and the Agrobacterium extracellular polysaccharide are not added; and CRₜₛ is the change rate of the absorbance of acetylcholinesterase at 412 nm when the cationic hydroxyethyl cellulose and the Agrobacterium extracellular polysaccharide are added.

The Agrobacterium extracellular polysaccharide and the cationic hydroxyethyl cellulose prepared in Example 1-3 were detected by using the above method, respectively. As shown in FIG. 6, results show that when the concentration of the Agrobacterium extracellular polysaccharide is 0.1-1 g/L, the relative activity of acetylcholinesterase is not decreased; and when the concentration of the cationic hydroxyethyl cellulose is 10 g/L, the relative activity of acetylcholinesterase is 80.6%. In general, a substance at a content level that causes 50% loss of the activity of acetylcholinesterase is considered to be toxic. Obviously, the Agrobacterium extracellular polysaccharide and the cationic hydroxyethyl cellulose prepared in the present invention have no obvious toxicity to the acetylcholinesterase within use concentrations.

### Application Example 1

Inhibitory effect of cationic hydroxyethyl cellulose on Cyanobacteria

(1) A Microcystis solution with an OD₆₀₀ of 0.1 was formulated, 18 pieces of 75 mL breathable flasks were selected, 30 mL of the algae solution was loaded into each breathable flask, every three breathable flasks were used as a group, and the breathable flasks were numbered as 7-0, 7-0, 7-0, 7-1, 7-1, 7-1, . . . , 7-6, 7-6, 7-6. The concentration of cationic hydroxyethyl cellulose in the breathable flasks loaded with the algae solution was 0 ppm, 8 ppm, 10 ppm, 13 ppm, 20 ppm and 40 ppm, with three parallel concentrations in each group.

(2) The breathable flasks in the step (1) were cultured at a constant temperature of 25° C. under moderate light for 72 h.

(3) Determination of the chlorophyll content of Cyanobacteria in the breathable flasks in the step (2): 1) Appropriate quantities of 0.1 mm zirconium beads and 0.5 mm zirconium beads were added into a 2 mL cryopreservation tube; 2) 3 mL of the algae solution was subjected to suction filtration under vacuum to obtain algae mud, the algae mud was placed in the cryopreservation tube with the zirconium beads, and then 1.5 mL of 90% acetone was added; 3) the cryopreservation tube was crushed in cycles for 5 times in a cell crusher according to an operation procedure of crushing for 10 s and stopping for 10 s; and 4) the cryopreservation tube was taken out, refrigerated at 4° C. for 2 h and centrifuged to obtain a supernatant, the wavelength of the supernatant at 630 nm, 647 nm, 664 nm and 750 nm was tested, and the chlorophyll content and the chlorophyll clearance rate were finally calculated according to the following calculation formula.

$$Chlorophyll content ( μg / L ) = \frac{11.85 \times ( {A}_{664} - {A}_{750} ) - 1.54 \times ( {A}_{647} - {A}_{750} ) - 0.08 \times ( {A}_{630} - {A}_{750} )}{2}$$

$$Chlorophyll clearance rate ( \% ) = \frac{{ChlA}_{1} - {ChlA}_{2}}{{ChlA}_{1}} \times 100 \%$$

In the formula, A₆₆₄ is the absorbance at 664 nm, A₇₅₀ is the absorbance at 750 nm, A₆₄₇ is the absorbance at 647 nm, and A₆₃₀ is the absorbance at 630 nm. ChlA₁ is the chlorophyll content of the algae solution before adding the cationic hydroxyethyl cellulose, and ChlA₂ is the chlorophyll content of the algae solution after adding the cationic hydroxyethyl cellulose for 72 h.

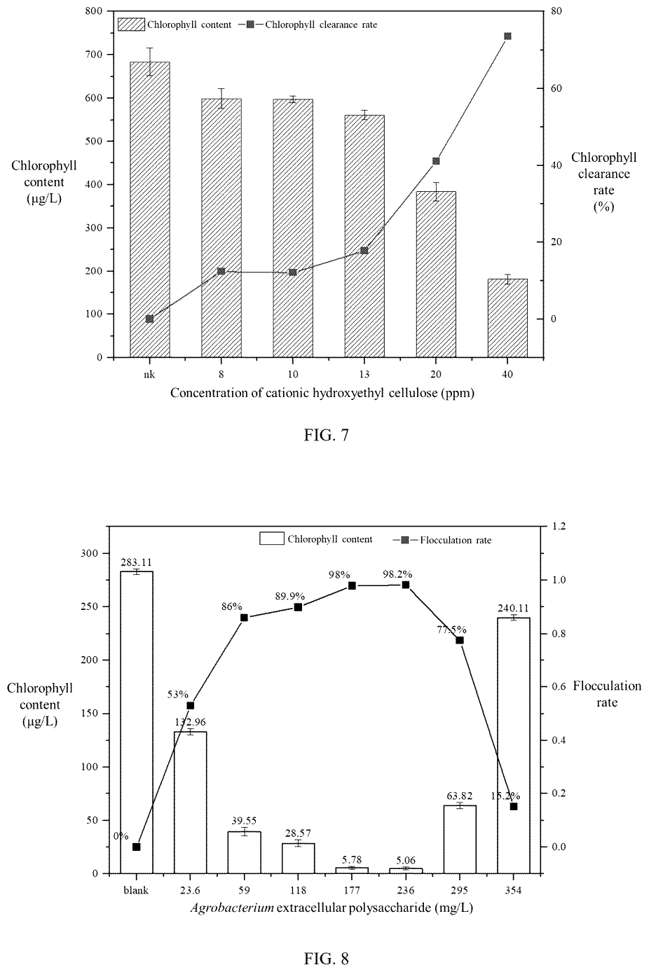

Results are shown in FIG. 7. The results show that when the concentration of the cationic hydroxyethyl cellulose is 40 ppm, the chlorophyll clearance rate of Microcystis aeruginosa reaches 73%, indicating that the cationic hydroxyethyl cellulose has an inhibitory effect on the Cyanobacteria.

### Application Example 2

Flocculation effect of a biological flocculant on Cyanobacteria

(1) 600 mL of a Microcystis solution with an OD₆₀₀ of 0.1 was selected, separately loaded into 50 mL to 100 mL beakers, and magnetically stirred evenly.

(2) 50 mL of the Microcystis solution in a logarithmic phase was selected and placed in a 100 mL small beaker, certain concentrations of cationic hydroxyethyl cellulose and Agrobacterium extracellular polysaccharide were added, stirring was performed on a magnetic stirrer for 1 min, and then standing was performed for 1 min. After the standing was performed, 3 mL of the algae solution was sucked from a same position at the bottom of the solution with a pipette gun. The chlorophyll content was determined (a test method was the same as that in Application Example 1), and the flocculation rate of Cyanobacteria after adding a biological flocculant was calculated based on the loss rate of the chlorophyll content of the algae solution. 11 concentration gradients of the cationic hydroxyethyl cellulose were set as 10 mg/L, 20 mg/L, 25 mg/L, 50 mg/L, 100 mg/L, 200 mg/L, 250 mg/L, 300 mg/L, 400 mg/L, 500 mg/L, and 600 mg/L, respectively. 7 concentration gradients of the Agrobacterium extracellular polysaccharide were set as 23.6 mg/L, 59 mg/L, 118 g/L, 177 mg/L, 236 mg/L, 295 mg/L, and 354 mg/L, respectively.

(3) The flocculation rate was calculated according to the following formula:

$$Flocculation rate ( \% ) = \frac{{ChlA}_{3} - {ChlA}_{4}}{{ChlA}_{3}} \times 100 \%$$

In the formula, ChlA₃ is the chlorophyll content of the algae solution before flocculation, and ChlA₄ is the chlorophyll content of the algae solution after flocculation.

Results are shown in Table 1 below. Considering the cost and the safety comprehensively, when the concentration of the cationic hydroxyethyl cellulose is 20 mg/L and the concentration of the Agrobacterium extracellular polysaccharide is 236 mg/L, the flocculation rate is 98.2%. When the concentration of the cationic hydroxyethyl cellulose is 20 mg/L and the concentration of the Agrobacterium extracellular polysaccharide is 177 mg/L, the flocculation rate is 99.8%. The latter effect is better.

TABLE 1

| | Cationic | Agrobacterium | |
|---|---|---|---|
| | hydroxyethyl | extracellular | Flocculation |
| Example | cellulose (mg/L) | polysaccharide (mg/L) | rate (%) |
| 1-1 | 20 | 23.6 | 53.0 |
| 1-2 | 20 | 59 | 86.0 |
| 1-3 | 20 | 118 | 89.9 |
| 1-4 | 20 | 177 | 98.0 |
| 1-5 | 20 | 236 | 98.2 |
| 1-6 | 20 | 295 | 77.5 |
| 1-7 | 20 | 354 | 15.2 |
| 2-1 | 10 | 177 | 35.2 |
| 2-2 | 20 | 177 | 99.8 |
| 2-3 | 25 | 177 | 95.7 |
| 2-4 | 50 | 177 | 82.2 |
| 2-5 | 100 | 177 | 81.9 |
| 2-6 | 200 | 177 | 73.5 |
| 2-7 | 250 | 177 | 73.0 |
| 2-8 | 300 | 177 | 66.1 |
| 2-9 | 400 | 177 | 56.9 |
| 2-10 | 500 | 177 | 56.6 |
| 2-11 | 600 | 177 | 42.6 |

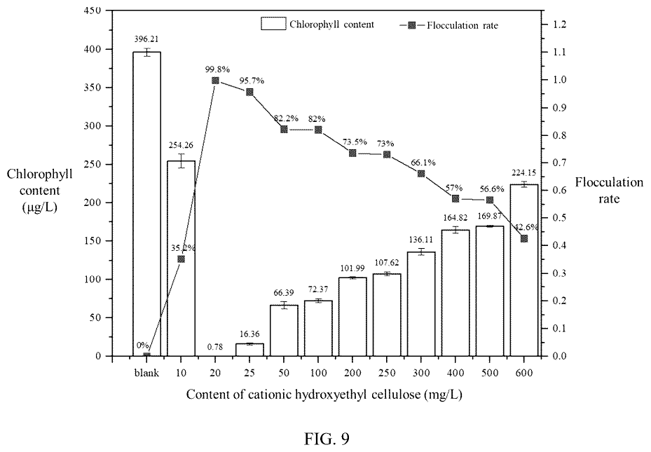

FIG. 8 shows flocculation rate results of Cyanobacteria at different contents of Agrobacterium extracellular polysaccharides when the content of the cationic hydroxyethyl cellulose is 250 g/L. When the concentration of the Agrobacterium extracellular polysaccharide is 23.6-236 mg/L, the flocculation rate of the Cyanobacteria is increased from 53.0% to 98.2%. When the concentration of the Agrobacterium extracellular polysaccharide is 236-354 mg/L, the flocculation rate of the Cyanobacteria is decreased from 98.2% to 15.2%. FIG. 9 shows flocculation rate results of Cyanobacteria at different contents of cationic hydroxyethyl cellulose when the content of the Agrobacterium extracellular polysaccharide is 177 mg/L. When the concentration of the cationic hydroxyethyl cellulose is 10-20 mg/L, the flocculation rate of the Cyanobacteria is increased from 35.2% to 99.8%. When the concentration of the cationic hydroxyethyl cellulose is 20-600 mg/L, the flocculation rate of the Cyanobacteria is decreased from 99.8% to 42.6%. It is indicated that when the content of the cationic hydroxyethyl cellulose or the Agrobacterium extracellular polysaccharide in the Cyanobacteria algae solution is too high (or too low), charge neutralization in the Cyanobacteria algae solution is not complete, finally leading to decrease of the flocculation rate.

The descriptions above are only preferred embodiments of the present invention. It should be noted that for those of ordinary skill in the technical field, various improvements can also be made without departing from the principles of the present invention, and all the improvements should also be regarded as falling within the scope of protection of the present invention.

1. A biological flocculant, comprising cationic hydroxyethyl cellulose and an extracellular polysaccharide; and the extracellular polysaccharide is produced by fermentation of Agrobacterium sp. LHZJX.A07 that is taxonomically named, the strain has been preserved in Guangdong Microbial Culture Collection Center with a accession number GDMCC NO: 62125 on a preservation date of Mar. 17, 2022, and an address of a preservation department is Institute of Microbiology, Guangdong Academy of Sciences, 5th Floor, No. 59 Building, No. 100 Courtyard, Martyr Middle Road, Guangzhou.

2. A method for preparing the biological flocculant according to claim 1, comprising the following steps:
(1) isolating and screening an Agrobacterium strain from soil, purifying and inoculating the strain into a slant culture medium for storage; and rinsing off bacterial moss on a slant with sterile water under sterile conditions, and inoculating a bacterial solution into a seed culture medium for culture;
(2) inoculating a seed solution cultured in the step (1) into a fermentation culture medium for culture;
(3) subjecting a fermentation solution in the step (2) to centrifugation, and collecting a supernatant to obtain a extracellular polysaccharide crude solution;
(4) subjecting the extracellular polysaccharide crude solution obtained in the step (3) to concentration, purification and drying to obtain an extracellular polysaccharide;
(5) dispersing hydroxyethyl cellulose in a mixture of isopropanol and water, performing stirring evenly, and then adding a 25 wt % NaOH solution and a 65 wt % 3-chloro-2-hydroxypropyltrimethylammonium chloride solution to carry out a reaction; and after the reaction is completed, performing purification with acetone, and performing washing, freeze drying and sieving to obtain cationic hydroxyethyl cellulose; and
(6) evenly stirring the cationic hydroxyethyl cellulose prepared in the step (5), then adding the Agrobacterium extracellular polysaccharide prepared in the step (4), performing stirring evenly, and then performing standing to obtain the biological flocculant.

3. The method for preparing the biological flocculant according to claim 2, wherein a formulation of the seed culture medium in the step (1) comprises: sucrose (4-6 g/L), Na₂HPO₄ (1-4 g/L), MgSO₄·7H₂O (0.2-1 g/L), CaCO₃ (0.05-0.15 g/L) and FeCl₃ (0.001-0.01 g/L), and has a pH of 6.5-7.5.

4. The method for preparing the biological flocculant according to claim 2, wherein a formulation of the fermentation culture medium in the step (2) comprises: KH₂PO₄ (0.1-0.5 g/L), MgSO₄·7H₂O (0.1-0.5 g/L), CaCO₃ (2-5 g/L), CaSO₄ (0.05-0.2 g/L), NaCl (0.1-0.5 g/L) and mannitol (8-10 g/L), and has a pH of 6.8-7.0.

5. The method for preparing the biological flocculant according to claim 2, wherein conditions for inoculating the bacterial solution into the seed culture medium for culture in the step (1) comprise: culture at 30-40° C. and 125-150 r/min for 12-24 h; and conditions for inoculating the seed solution into the fermentation culture medium for culture in the step (2) comprise: culture at 30-40° C. and 120-150 r/min for 1-5 d.

6. The method for preparing the biological flocculant according to claim 2, wherein the subjecting the extracellular polysaccharide crude solution to concentration, purification and drying in the step (4) specifically comprises the following steps:
(a) subjecting the extracellular polysaccharide crude solution to concentration by using a dialysis bag to obtain an extracellular polysaccharide concentrate; and adding the extracellular polysaccharide concentrate into ethanol, performing stirring at a constant speed by using a magnetic stirrer for 5-10 min, performing standing for 12-24 h, performing centrifugation at 1×10⁴-1.6×10⁴ r/min, removing a supernatant, and collecting a precipitate;
(b) adding pure water that is ⅓ of the volume of the extracellular polysaccharide concentrate in the step (a) into the precipitate collected in the step (a), performing dissolution in a water bath at 37-45° C. for 20-60 min, and performing centrifugation to collect a supernatant; and
(c) adding the supernatant collected in the step (b) into ethanol, performing stirring at a constant speed by using a magnetic stirrer for 5-10 min, performing standing for 12-24 h, performing centrifugation at 1×10⁴1.6×10⁴ r/min, removing a supernatant, collecting a precipitate, and performing freeze drying and sieving with a 60-100 mesh sieve to obtain the Agrobacterium extracellular polysaccharide.

7. The method for preparing the biological flocculant according to claim 6, wherein a cut-off amount of the dialysis bag used in the step (a) is 3-8 kDa (kilodalton), and a dialysis time is 48-72 h; a temperature of the freeze drying in the step (c) is 0-4° C.; a mass fraction of the ethanol in the step (a) and the step (c) is 95%-100%; and a volume ratio of the extracellular polysaccharide concentrate to the ethanol in the step (a) is 1:2 to 1:6, and a volume ratio of the supernatant to the ethanol in the step (c) is 1:2 to 1:6.

8. The method for preparing the biological flocculant according to claim 2, wherein an addition amount of the 25 wt % NaOH solution in the step (5) is 3-8 mL, an addition amount of the 65 wt % 3-chloro-2-hydroxypropyltrimethylammonium chloride solution is 3-10 mL, and a mesh number of the sieving is 60-100.

9. The method for preparing the biological flocculant according to claim 2, wherein a temperature of the reaction in the step (5) is 60-70° C., and a time of the reaction is 2-8 h.

10. Use of a biological flocculant obtained by the preparation method according to claim 2 in control of Cyanobacteria.

11. The use according to claim 10, wherein a formulation of the seed culture medium in the step (1) comprises: sucrose (46 g/L), Na₂HPO₄ (1-4 g/L), MgSO₄·7H₂O (0.2-1 g/L), CaCO₃ (0.05-0.15 g/L) and FeCl₃ (0.001-0.01 g/L), and has a pH of 6.5-7.5.

12. The use according to claim 10, wherein a formulation of the fermentation culture medium in the step (2) comprises: KH₂PO₄ (0.1-0.5 g/L), MgSO₄-7H₂O (0.1-0.5 g/L), CaCO₃ (2-5 g/L), CaSO₄ (0.05-0.2 g/L), NaCl (0.1-0.5 g/L) and mannitol (8-10 g/L), and has a pH of 6.8-7.0.

13. The use according to claim 10, wherein conditions for inoculating the bacterial solution into the seed culture medium for culture in the step (1) comprise: culture at 30-40° C. and 125-150 r/min for 12-24 h; and conditions for inoculating the seed solution into the fermentation culture medium for culture in the step (2) comprise: culture at 30-40° C. and 120-150 r/min for 1-5 d.

14. The use according to claim 10, wherein the subjecting the extracellular polysaccharide crude solution to concentration, purification and drying in the step (4) specifically comprises the following steps:
(a) subjecting the extracellular polysaccharide crude solution to concentration by using a dialysis bag to obtain an extracellular polysaccharide concentrate; and adding the extracellular polysaccharide concentrate into ethanol, performing stirring at a constant speed by using a magnetic stirrer for 5-10 min, performing standing for 12-24 h, performing centrifugation at 1×10⁴-1.6×10⁴ r/min, removing a supernatant, and collecting a precipitate;
(b) adding pure water that is ⅓ of the volume of the extracellular polysaccharide concentrate in the step (a) into the precipitate collected in the step (a), performing dissolution in a water bath at 37-45° C. for 20-60 min, and performing centrifugation to collect a supernatant; and
(c) adding the supernatant collected in the step (b) into ethanol, performing stirring at a constant speed by using a magnetic stirrer for 5-10 min, performing standing for 12-24 h, performing centrifugation at 1×10⁴-1.6×10⁴ r/min, removing a supernatant, collecting a precipitate, and performing freeze drying and sieving with a 60-100 mesh sieve to obtain the Agrobacterium extracellular polysaccharide.

15. The use according to claim 10, wherein a cut-off amount of the dialysis bag used in the step (a) is 3-8 kDa, and a dialysis time is 48-72 h; a temperature of the freeze drying in the step (c) is 0-4° C.; a mass fraction of the ethanol in the step (a) and the step (c) is 95%-100%; and a volume ratio of the extracellular polysaccharide concentrate to the ethanol in the step (a) is 1:2 to 1:6, and a volume ratio of the supernatant to the ethanol in the step (c) is 1:2 to 1:6.

16. The use according to claim 10, wherein an addition amount of the 25 wt % NaOH solution in the step (5) is 3-8 mL, an addition amount of the 65 wt % 3-chloro-2-hydroxypropyltrimethylammonium chloride solution is 3-10 mL, and a mesh number of the sieving is 60-100.

17. The use according to claim 10, wherein a temperature of the reaction in the step (5) is 60-70° C., and a time of the reaction is 2-8 h.


SEQUENCE LISTING (1 sequence)

SEQ ID NO: 1 - DNA, 1341 bp, Agrobacterium sp.
  source 1..1341: mol_type = genomic DNA
ccccgcaaggggagtggcagacgggtgagtaacgcgtgggaacataccctttcctgcggaatagctccgggaaactggaattaataccgcatacgccctacgggggaaagatttatcggggaaggattggcccgcgttggattagctagttggtggggtaaaggcctaccaaggcgacgatccatagctggtctgagaggatgatcagccacattgggactgagacacggcccaaactcctacgggaggcagcagtggggaatattggacaatgggcgcaagcctgatccagccatgccgcgtgagtgatgaaggccttagggttgtaaagctctttcaccggagaagataatgacggtatccggagaagaagccccggctaacttcgtgccagcagccgcggtaatacgaagggggctagcgttgttcggaattactgggcgtaaagcgcacgtaggcggatatttaagtcaggggtgaaatcccagagctcaactctggaactgcctttgatactgggtatcttgagtatggaagaggtaagtggaattccgagtgtagaggtgaaattcgtagatattcggaggaacaccagtggcgaaggcggcttactggtccattactgacgctgaggtgcgaaagcgtggggagcaaacaggattagataccctggtagtccacgccgtaaacgatgaatgttagccgtcgggcagtatactgttcggtggcgcagctaacgcattaaacattccgcctggggagtacggtcgcaagattaaaactcaaaggaattgacgggggcccgcacaagcggtggagcatgtggtttaattcgaagcaacgcgcagaaccttaccagctcttgacattcggggtatgggcattggagacgatgtccttcagttaggctggccccagaacaggtgctgcatggctgtcgtcagctcgtgtcgtgagatgttgggttaagtcccgcaacgagcgcaaccctcgcccttagttgccagcatttagttgggcactctaaggggactgccggtgataagccgagaggaaggtggggatgacgtcaagtcctcatggcccttacgggctgggctacacacgtgctacaatggtggtgacagtgggcagcgagacagcgatgtcgagctaatctccaaaagccatctcagttcggattgcactctgcaactcgagtgcatgaagttggaatcgctagtaatcgcagatcagcatgctgcggtgaatacgttcccgggccttgtacacaccgcccgtcacaccatgggagttggttttacccgaaggtagtgcgctaaccgcaaggaggcagctaac